(19) United States
(12) Patent Application Publication
(10) Pub. No.: US 2026/0042987 A1
(43) Pub. Date: Feb. 12, 2026

(54) PROCESS FOR T CELL EXPANSION

(71) Applicants: AlloVir, Inc., Waltham, MA (US); Baylor College of Medicine, Houston, TX (US); Wilson Wolf Manufacturing, New Brighton, MN (US)

(72) Inventors: Rainer Ludwig Knaus, London (GB); Katy Rebecca Newton, London (GB); Juan Vera, Houston, TX (US); Ann Leen, Houston, TX (US); Cliona Rooney, Bellaire, TX (US); John R. Wilson, New Brighton, MN (US)

(73) Assignees: AlloVir, Inc., Waltham, MA (US); Baylor College of Medicine, Houston, TX (US); Wilson Wolf Manufacturing, New Brighton, MN (US)

(21) Appl. No.: 19/361,743

(22) Filed: Oct. 17, 2025

Related U.S. Application Data
(63) Continuation of application No. 17/586,698, filed on Jan. 27, 2022.
Application No. 17/586,698 is a continuation of application No. 16/016,305, filed on Jun. 22, 2018, now Pat. No. 11,963,979.
Application No. 16/016,305 is a continuation of application No. 14/364,621, now abandoned.

(30) Foreign Application Priority Data
Dec. 12, 2011 (GB) 1121308.9

(51) Int. Cl. C12M 1/04; A61K 39/00; A61K 39/12; A61K 39/235; A61K 39/245; A61K 40/11; A61K 40/46; C12N 5/0783; C12N 7/00

(52) U.S. Cl. CPC C12M 23/24; A61K 39/12; A61K 39/235; A61K 39/245; A61K 40/11; A61K 40/46; C12N 5/0636; C12N 7/00; A61K 2039/55527; A61K 2239/31; A61K 2239/38; C12N 2501/2304; C12N 2501/2307; C12N 2506/11; C12N 2710/10034; C12N 2710/16134; C12N 2710/18034; Y02A 50/30

(57) ABSTRACT

An in vitro expansion process for rapid expansion of antigen specific T cells, such as allogeneic antigen specific T cells comprising the steps culturing in a gas permeable vessel a population of PBMCs (such as allogeneic PBMCs) in the presence of antigen, for example a peptide or peptide mix relevant to a target antigen(s), in the presence of an exogenous cytokine characterized in that the expansion to provide the desired population of T cells is 14 days or less, or example 9, 10, 11 or 12 days, such as 10 days. The disclosure also extends to T cell populations generated by and obtained from the method and the use of same in therapy.

### CROSS-REFERENCE TO RELATED APPLICATIONS

This application is continuation of U.S. Non-Provisional patent application Ser. No. 17/586,698 filed on Jan. 27, 2022, which is a continuation of U.S. Non-Provisional patent application Ser. No. 16/016,305 filed on Jun. 22, 2018, issued as U.S. Pat. No. 11,963,979 on Apr. 23, 2024, which is a continuation of U.S. Non-Provisional patent application Ser. No. 14/364,621 filed on Jun. 11, 2014 and now abandoned, which is a national phase application under 35 U.S.C. § 371 that claims priority to International Application No. PCT/GB2012/053113 filed Dec. 12, 2012, which claims priority to Great Britain Patent Application No. 1121308.9 filed Dec. 12, 2011; all of which are incorporated herein by reference in their entirety.

The present invention relates to a novel process for expanding T cells, in particular antigen specific T cells, such as allogeneic T cells, cell populations therefrom, pharmaceutical compositions comprising the said cell populations and use of the cells and compositions for treatment, particular the treatment of prophylaxis of virus infection, especially in immune compromised patients.

### BACKGROUND

Immune compromised patients are susceptible to opportunistic virus infection. This is a huge problem in bone marrow transplant patients because their immune cells are routinely depleted as part of the bone marrow transplant procedure and other times rendered non-functional due to steroid treatment for Graft versus Host Disease (GvHD) which is a common complication of bone marrow transplantation. Latent viruses such as Cytomegalovirus (CMV) and Adenoviruses (ADV) become re-activated and the body is unable to fight the infection. The term bone marrow transplantation as used herein describes all forms of allogeneic haematopoietic stem cell transplantation (allo-HSCT) including procedures involving stem cell donation from related or unrelated donors or from cord blood.

A practice of immune reconstitution has developed and this involves the transplant (adoptive transfer) into the transplant patient of immune cells from a matched HLA donor, usually the same donor who provided the bone marrow. These cells appear to engraft in the patient to provide long-term immunity to pathogens or at least interim assistance in fighting infection until the patients own immune system is fully reconstituted through the engraftment of the donor's haematopoietic stem cells which will then develop into a diverse array of blood cells and immune cells.

Years of clinical research into the adoptive transfer of donor immune cells to achieve immune reconstitution in a patient following a bone marrow transplant has illustrated the benefits of this approach as well as the challenges of optimising the approach to ensure a consistently efficacious an safe result. In some cases, the number of donor immune cells which are necessary to effect immune reconstitution against a specific pathogen cannot be obtained through simple mechanical selection systems. In such cases, the minimum dosing of the therapeutic immune cells, in particular antigen-specific T cells which demonstrate an adaptive memory immune response against the target pathogen, can be obtained by expanding the desired donor T cell population on an ex vivo basis using a cell culture system. Prior art indicates that the process of expansion of the celts from the donor sample generally takes about 21 days and the focus has been to expand the specific cells in order to obtain the highest possible number (yield) of the relevant cell population as well as the highest possible purity of the relevant cell populations, so for example to obtain a population which is as close to 100 percent pure for the target cells. This obviously takes long periods of expansion and repeated antigen stimulation to ensure the cells keep expanding, through cell culture. Whilst not wishing to be bound by theory, it is likely that the thinking behind this was two-fold, firstly the larger the number of relevant cells the more effective the treatment will be and secondly by ensuring a highly purified target population toxicities and unwanted effects from contaminating cell products are avoided.

Exogenous IL-2 has traditionally been employed in the culture since this cytokine has been characterised as a T cell growth factor. Generally, the source of exogenous of IL-2 is required once or twice a week during the expansion process.

The inventors have established a rapid process for the expansion of antigen-specific T cells that provides one or more of the following advantages in that it is:
1) efficient, robust, viable, and/or economically cost-effective for producing the cell product;
2) provides a therapeutic dose (yield) of the antigen-specific T cells within a minimum period of culture time;
3) minimises contamination from other cells which may cause unwanted toxicities in the patient, in particular Graft vs Host Disease in a clinically relevant context;
4) ensures compliance with GMP production requirements through the development of new and inventive technological adaptions to existing technology which does not conform to GMP requirements;
5) provides the option to omit the addition of exogenous IL-2;
6) allows expansion from a donor blood sample which is considered mobilised as defined by the donor having received G CSF prior to making the bone marrow (haematopoietic stem cell) donation;
7) provides a product with equivalent or improved characteristics over the prior art by optimising a desirable balance be reached between the adequate expansion of antigen-specific T cell populations and the presence of potentially harmful contaminating cell populations; and
8) allows expansion from a small donor sample in the range 50-100 mls.

This will be explained in more detail below.

In addition to those practical improvements the present inventors have reason to believe that the prior art methods of culturing cells for prolonged periods particularly in the presence of IL-2 may result in T cell populations which demonstrate a certain degree of exhaustion or energy. That is to say there may be a large number of the desired cells present in the population but many of these may not be functioning or may be functioning sub-optimally, for example hyporesponsive in one more or more functional aspects.

### SUMMARY OF THE INVENTION

The present inventors have devised an in vitro expansion process for rapid expansion of antigen specific T cells, such as allogeneic antigen specific T cells comprising the steps culturing, in a vessel comprising a gas permeable culture surface, a population of PBMCs (in particular allogeneic PBMCs) in the presence of a peptide or peptide mix relevant to a target antigen(s), and in the presence of at least one exogenous cytokine characterised in that the at least one cytokine is other than exogenous IL-2.

There is also provided an in vitro expansion process for rapid expansion of allogeneic antigen specific T cells comprising the steps of culturing, in a vessel comprising a gas permeable culture surface, a population of allogeneic PBMCs in the presence of a peptide or peptide mix relevant to a target antigen(s), in the presence of at least one exogenous cytokine characterised in that the expansion to provide the desired population of antigen specific T cells is performed for 14 days or less.

The process of the present disclosure has many advantages in that it reduces the time and resources required to expand the antigen-specific T cells, it is robust and minimises the risk of contamination and these aspects are of huge practical significance because the process can be made GMP compliant and will make the therapy accessible to a larger number of patients in reed thereof.

### BRIEF DESCRIPTION OF THE DRAWINGS

FIG. 1 is a diagrammatic representation of the GRex system available from Wilson Wolf.

FIG. 2 is a diagrammatic representation of apparatus employed in FIG. 3.

FIG. 3 is a diagrammatic representation of a modified GRex system suitable for use as a closed system.

FIGS. 4A & B depict a graph of two samples showing that 50-100 ml of blood draw is sufficient to generate a sufficient dose of adenovirus specific T cells grown using IL-4/7 but not without.

FIGS. 5A & B show the spot forming cells in a cytokine specific assay for expanded cells from donor II and Ill which were cultured in the presence of CMV peptide pp65, IL4 and IL7 or in the absence of cytokines.

FIGS. 6A & 6B show the cell growth rates for CMV and adeno specific expanded populations.

FIG. 7 shows adenovirus and CMV specific cytokine production using Elispot.

FIG. 8 IL4 expansion protocol vs IL2 expansion protocol.

FIG. 9 Culture with peptide concentrations of 5 ng/ml (CMV), 50 ng/ml (10×CMV) or 250 ng/ml (50×CMV).

FIG. 10 shows IFNg productions at different peptide concentrations.

### DETAILED DESCRIPTION OF THE INVENTION

Rapid expansion as employed herein refers to a process in a therapeutic product is obtained within less than 18 days, such as 7-10 days.

The antigen specific T cell population obtained from the process is at least equivalent to the product obtained in the prior art methods but may in a number of respects may have improved properties, for example they may exhibit less or zero energy or exhaustion in comparison to cells prepared by prior art processes.

The activation of T cells is tightly controlled by many positive and negative regulatory processes. This allows the body to provide immunity to pathogens whilst minimising autoimmunity. Activation of T cells occurs when specific molecules on the surface of professional antigen presenting cells such as dendritic cells bind the T cell receptor and also provide a co-stimulatory factor.

It is documented that anergy or hyporesponsiveness can occur, for example when antigen on antigen presenting cells engages the T cell receptor in the absence of a co-stimulatory signal. This hyporesponsiveness may be manifest by reduced levels of cytokine secretion, for example lower levels of interferon-gamma secretion and/or lower levels of IL-13 secretion.

The lack of anergy, for example may be illustrated in that cells prepared by the present process on average (mean) secrete more interferon gamma than cells prepared by the prior art process, when the secretion of the population as whole is measured and then divided by the number of cells in the relevant population to provide the mean.

In vitro at least certain types of anergy are reversible, for example by the addition of IL-2. In vitro it is not known how anergic cells respond in the case of adoptive transfer of cells to a patient whether anergic cells can be revived to perform their natural functions.

Whilst not wishing to be bound by theory when preparing therapeutic antigen specific T cell populations, the absolute number of cells for dosing may not be the most definitive factor in providing an effective product. Rather the functionality of the cells administered may be more relevant. That is to say administering cells wherein the number of hyporesponsive cells are minimised.

The present process optimises the amount of functional cells in that the culturing of the cells is for a relatively short period, generally 14 days or less. Thus the cells are not artificially activated to the point where they start to become unviable or hyporesponsive.

In one embodiment the cells are expanded in the absence of exogenous IL-2 and instead in the presence of only endogenous IL-2. Although IL-2 is known to be a T cell stimulator, the empirical observation when employed in the present method is that it seems to drive proliferation too fast and may result in some disadvantages.

In one embodiment a small amount of exogenous IL-2 is employed, e.g. 10 units or less per ml.

An exogenous factor is one that is not present in the culture of PBMCs without addition or where the naturally occurring amounts present in the cell culture are augmented by addition of exogenous amounts of the factor.

Expanding the cells for a reduced period of time and/or without the addition of exogenous IL-2 means that the artificial conditions (for example sustained periods of expansion and/or high levels of stimulation) of the prior art process are avoided and thus anergy/exhaustion resulting from the same may be minimised.

The current process balances generating a sufficient population of antigen-specific T cells whilst minimising but not necessarily eliminating the non-target populations of cells.

Generally the expanded cell population obtained from the current process does not contain more than 80% of the target antigen specific T cell population and this is a marked departure from the established wisdom of the field.

Whilst not wishing to be bound by theory it is believed that the population of antigen-specific T cells generated is sufficient to continue expanding in vivo. This may be in part because the cells produced are not anergic/exhausted.

The cells administered to the patient by infusion are intended to continue expanding in vivo. It is believed that antigen-specific T cells generated by the process described herein are suitable for the intended therapeutic purpose.

Other cell populations in the expanded product according to the present disclosure are at least not harmful and may in fact be beneficial, for example because the overall population of cells infused into the patient may be more representative of the natural environment in vivo and thus more compatible therewith.

Anergy in the context of the present specification is intended to refer to a T cells' functional unresponsiveness in one or more ways in the presence of antigen, for example failure to undergo antigen-specific expansion and/or failure to secrete cytokines such as TNF-α or interferon-gamma.

Populations, as employed herein comprise a number of individual cells. The relevant population will not be considered anergic to the extent that at least 80% of the population are not anergic in any respect, for example 85, 90, 95 or 100% of the relevant cells are not anergic. Of course anergy is not a binary analysis but should be viewed within a range of the quality of T cell responses as measured by one or more functional assays.

As described above anergy in the context of the present specifications is intended to be a generic term that refers to reduced cell function in one or more relevant ways. The term includes cell exhaustion, for example where the cells are no longer able to divide. The cell is then referred to as senescent. Cells stop dividing because the telomeres, protective lengths of DNA on the end of a chromosome required for replication, shorten with each cell division, eventually being reduced to a point which through biological mechanisms limits further cell division.

In one embodiment the energy is hyporesponsiveness.

In one embodiment anergy may be reversible in vitro by addition of IL-2. This feature may be employed as an assay for certain types of anergy.

There are certain cell surface markers that are exhibited by anergic cells, for example PD-1 (programmed cell death protein 1 Uniprot Q15116) or PD-1 ligand. In one embodiment 10% or less such as 9, 8, 7, 6, 5, 4, 3, 2 or 1% of the expanded antigen specific T cell population express PD-1 on their surface. Blimp-1 may be another cell surface marker of anergy.

In another embodiment, PD-1 is not permanently expressed on the T cells and expression is reversible Markers for cells that may apoptotic may include down regulation of one or more of CD4, CDS, HSA, CD45RB or a combination of the same and/or upregulation of one or more CD3/TCR, C069 and CD25). “T cell” is a term commonly employed in the art and intended to include all CD3+ cells including thymocytes, immature T lymphocytes, mature T lymphocytes, resting T lymphocytes or activated T lymphocytes. A T cell can be a T helper (Th) cell, for example a T helper 1 (Th1) or a T helper 2 (Th2) cell, although other grouping of T cell populations are being discovered based on intensive research. The T cell can be a CD4+ T cell CD8+ T cell, CD4+CD8+ T cell, CD4-CD8-T cell or any other subset of T cells.

The distinguishing feature of the T cells which are the product of the current process is that the T cell has a CD3+ associated T cell receptor (TCR) which provides the antigen-specificity which is a key aspect of the efficacy and safety of the cells for the intended purpose of reconstituting immunity on a pathogen-specific basis in the patient. The target population of cells can be selected employing this marker.

In one embodiment the target antigen specific T cell population is biased towards a CD4+ population. That is to say the antigen specific response is focused on the CD4+ cells. In one embodiment the populations contains more CD4+ cells than, for example CD8+ cells.

Antigen specific T ceil population as employed herein refers to T cells which are specific for the antigen which they target. Specific refers to the ability of the relevant T cells to discriminate against antigen targets and other entities to which they are not specific.

The vessel comprising a gas permeable culture surface is a significant part of the process because it allows efficient expansion in the presence of all the required nutrients (T cell expansion media), without the need to change the media or provide additional factors. Thus the vessel can support the volume of media required whilst allowing the gas exchange with the environment to ensure oxygen levels are sufficient for cell growth.

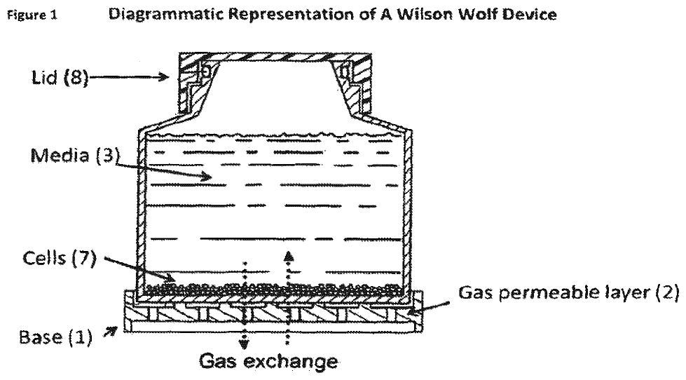

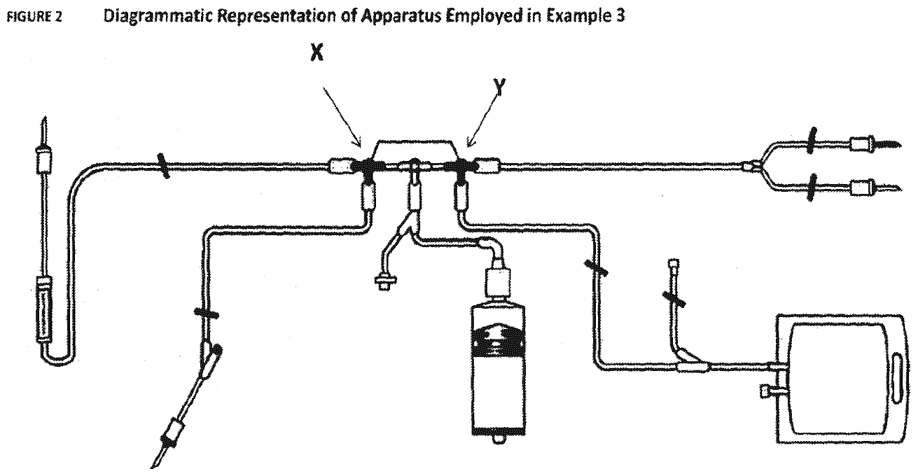

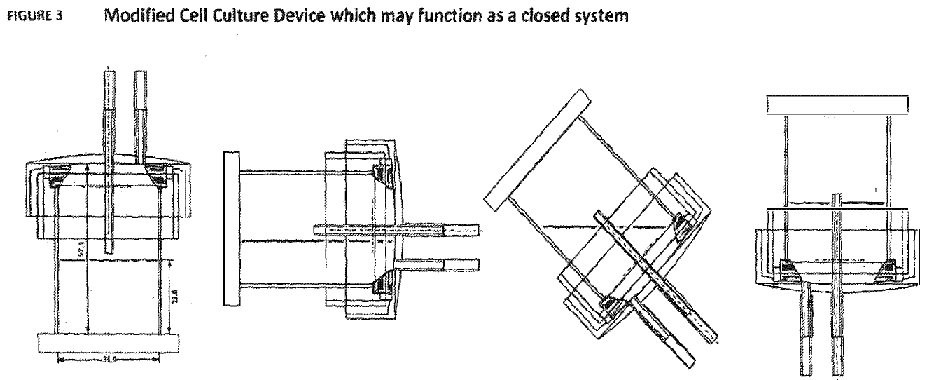

The process of the present disclosure suitably is performed in a vessel comprising a gas permeable culture surface. Vessel as employed herein is intended to refer to any type of container suitable for retaining the cells, media etc., for example a bag, such as an infusion type bag (provided with a gas permeable portion) or a rigid vessel, such as the GRex™ system see FIG. 1. The gas permeable culture surface facilitates rapid expansion of cells and minimizes the number of media changes required. This allows the manufacture of the expanded cells in a so-called one touch process, wherein all the components tor the process are inserted at the one time and can be left without interference until the expansion is complete. The only step that remains is to harvest the expanded cells.

WO 2005/035728 incorporated herein by reference describes how to prepare a gas permeable vessel (i.e., a vessel comprising a gas permeable culture surface). In one embodiment silicone gas permeable material is employed.

In one embodiment the system employed is a GRex™ system from Wilson Wolf. The system allows the cells to be expanded in 20 days or less, for example 14, 13, 12, 11, 10 days or less, such as 14 days or 10 days.

Rapid expansion as employed herein is expansion for about 18 days or less, such as 17, 16, 15, 14, 13, 12, 11, 10, 9, 8 or 7, such as 14, 13, 12, 11, 10, 9, 8 or 7 days. 9 or 10 days seems optimal in most cases.

This rapid expansion allows all the nutrients and media for the culture to be added, for example at the beginning of the process and the cells can grow and divide without any further intervention. This is advantageous because it minimizes contamination and potential for errors and also minimizes human resource required to expand the cells.

The system can also be adapted to render it a closed systems to allow the product to be manufactured aseptically, for example a described in U.S. provisional application Ser. No. 61/550,246, incorporated herein by reference. In one embodiment the system is a modified system as described in PCT/GB2012/052587 incorporated herein by reference.

Thus in one embodiment the system is a closed system suitable for the aseptic culturing therapeutic cells comprising:
(i) a vessel comprising:
   a gas permeable portion suitable for supporting cell growth and allowing delivery of gases to the cells during culturing, and
   at least one wall adjoined to a base,
   wherein said vessel defines an internal volume and said vessel is adapted to contain a requisite volume of medium to support a cell culture,
(ii) a vent comprising a conduit defining an interior orifice and on exterior orifice distal therefrom in fluid communication with each other, wherein the conduit extends from the exterior of the closed systems through a structural feature of the system and extends into the internal volume of the vessel and terminates therein with the interior orifice, wherein the interior orifice is arranged such that during filling and emptying of liquid medium it is not susceptible to blockage by liquid,
   wherein the exterior orifice is adapted to connect to an aseptic filter thereby allowing passage of gases through the filter into the vessel or out of the vessel, as required to achieve the entry and exit of fluids and cells into the vessel, and
(iii) a port or ports adapted to allow introduction of fluids and cells aseptically into the vessel, a port or ports adapted to allow fluids to exit the system without exposing the system to the external environment and adapted such that cells grown therein may exit the system under gravity when the system is orientated to put the ceils in fluid communication with the exit port and the latter is opened.

A system of this arrangement is shown in FIG. 3, by way of example.

In one embodiment the whole expansion process is performed aseptically in a closed system.

In one embodiment the system is seeded with about 0.5 to 2 million cells per cm² of surface area. In a GRex-10 with a surface area of 10 cm², a minimum of 5 million and up to 20 million cells would be seeded.

In one embodiment the system is seeded with about 20 million effector cells which equated to about 10 cm² of cells.

The present invention relates to ex vivo processing of cells and the T cell products obtained therefrom. Usually the present invention does not include the step of obtaining the sample from the patient.

The step of obtaining a suitable sample from the donor is a routine technique, which involves taking a blood sample. This process presents little risk to donors and does not need to be performed by a doctor but can be performed by appropriately trained support staff. In one embodiment the sample derived from the patient is approximately 200 ml of blood, or less, for example 50-100 ml.

Surprisingly sufficient numbers of antigen specific T cells can be generated using this small amount of blood.

Surprisingly the inventors have found that PBMCs from a mobilised blood sample may be employed in the expansion process. A mobilised sample is one where the donor has received G-CSF (granulocyte colony-stimulatory factor) and other factors which stimulate bone marrow to produce stem cells and then release them into the blood. Blood samples taken at this time have not been employed for later use in immune reconstitution therapy of the patient, simply because it was considered not suitable. Nawa et al in Bone Marrow Transplantation (2000) 25, 1035-1040, for example suggest that G-CSF results in reduced ability to secrete interferon gamma, IL-4 and also reduces proliferative responses. Shantaram et al in Blood, 15 Sep. 2001, Vol 98, number 6 also suggest that there is decreased immune functions of blood cells after mobilisation with G-CSF. Other research has suggested that G-CSF may skew the T cell population to the Th2 group, which may be less effective in controlling an intracellular viral infection.

However, it is inconvenient to require donors to return to hospital after haematopoietic stem cell donation to obtain further biological samples. In contrast it is very convenient to take a sample of blood for expansion to produce an immune reconstitution product at the time the as the stem cell sample for transplantation is taken from the donor.

Thus employing mobilised blood for expansion provides a practical advantage to health care workers and donors.

Mobilised blood as employed herein refers to a blood sample from a donor who has been mobilised by treatment with agent such as G-CSF. The process of mobilisation increases the number of sterns ceils in the peripheral blood.

Mobilised apheresis as employed herein refers to a sample from a donor who has been mobilised by treatment with agent such as G-CSF. The process of mobilisation increases the number of stems cells in the peripheral blood.

Typically the PBMCs for T cell expansion are obtained from the blood or apheresis product by Ficoll density gradient separation known to those skilled in the art.

As is known to the skilled person expansion of T cells is generally performed in a suitable T cell expansion media. T cell expansion media generally comprises serum, media and any cytokines employed in the expansion step, for example as specified in the consistory clause or claims, as appropriate.

In one embodiment the media is Advanced RPMI media or RPMI media 1640, available from Life Technologies. Advance RPMI media contains animal derived products and is usually employed with about 2% human serum. In contrast RPMI media 1640 does not contain animal derived products and is usually employed with 10% human serum. The RPMI media 1640 is generally more convenient for use in the present method.

Alternative serum free media is available from AQIX RS-I for lymphocyte culture.

In one embodiment the medium comprises 45% advanced RPMI, 45% EHAA, 10% FCs and 200 mM L-glutamine.

In one embodiment the cell expansion medium comprises 10% Human AB serum, 200 mM L-glutamine, 45% Earle's Ham's amino acids (EHAA or Click's medium) and 45% advanced RPMI or RPM1-1640.

In one embodiment the cytokines employed are discussed below.

In one embodiment the T cell expansion medium employed is not changed or supplemented during the expansion process.

Cell expansion as employed herein refers to increasing the number of the target cells in a population of cells as a result of cell division.

T cell expansion may be evaluated by counting viable CD3+ cells (i.e., the target population of cells is CD3+).

Viable cells can be tested by cell staining with Trypan blue (and light microscopy) or 7-amino-actinomycin D, vital dye omitting at 670 nm (or ViaProbe a commercial ready to use solution of 7AAD) and flow cytometry, employing a technique known to those skilled in the art. Where the stain penetrates into the cells the cells are considered not viable. Cells which do not take up dye are considered viable. An exemplary method may employ about 5 μL of 7AAD and about 5 μL of Annexin-V (a phospholipid binding protein which binds to external phospholipid phosphatidylserine exposed during apotosis) per approximate 100 μL of cells suspension. This mixture may be incubated at ambient temperature for about 15 minutes the absence of light. The analysis may then be performed employing flow cytometry. See for example MG Wing, AMP Montgomery, S. Songsivilai and JV Watson. An Improved Method for the Detection of Cell Surface Antigens in Samples of Low Viability using Flow Cytometry J Immunol Methods 126 21-27 1990.

An alternative stain is TO-PRO-3 which is a carbocyanine monomer nucleic acid stain with far-red fluorescence similar to Alexa Fluor 647 or Cy 5 dyes. It is useful as a nuclear counterstain and dead cell indicator, and is among the highest-sensitivity probes for nucleic acid detection.

Viruses against which antigen specific T cell populations can be expanded include cytomegalovirus, adenovirus, varicella zoster virus, BK virus, human papillomavirus, hepatitis B virus, hepatitis C virus, Epstein-Barr virus, Kaposi's sarcoma-associated herpes virus and human T-lymphotropic virus, such as cytomegalovirus or adenovirus.

Antigen employed in the process includes full-length polypeptides, fragments of polypeptides or peptides.

Peptide as employed herein is intended to refer to short polymers of amino acids linked by peptide bonds, wherein the peptides contain at least 2 but generally not more than 50 amino acids.

The peptides employed are sufficiently long to present one or more linear epitopes, for example are on average 7, 8, 9, 10, 11, 12, 13, 14, 15, 16, 17, 18, 19 or 20 amino acids long.

In one embodiment some of the peptides of the mixture overlap (in relation to the sequence of a single antigen), that is to say that they are from a single antigen end are arranged such that portions of the fragments and certain sequence of ammo acids from the parent sequence occur in more than one peptide fragment of the mix.

In one embodiment 1, 2, 3, 4, 5, 6, 7, 8, 9, 10, 11, 12, 13, 14 or 15 amino acids overlap in each peptide. In one embodiment the peptide libraries for each protein are 15 amino acids long and overlap by 11 amino acids so that all potential epitopes can be presented from a protein. The peptides can be longer, for example 20 amino acids overlapping by 15 or 30 amino acids overlapping by 25.

In one embodiment the largest virus is CMV and, for example the antigen employed to the target the virus is pp65. The sequence for human cytomegalovirus (strain AD169) is in the UniProt database under number PO6725. The recombinant protein can be purchased from Miltenyi Biotech. The latter company also provide PepTivator® CMV pp65 which is a peptide pool that consists mainly of 15-mer peptides with 11-amino acid (aa) overlap, covering the complete sequence of the pp65 protein of human cytomegalovirus, further target antigens for CMV include pp50 and IE-1 (also known as UL123). Target EBV antigens include EBNA1, LMP1, LMP2 and BARF1. Examples of suitable peptides sequences for these antigens include those in sequence ID NO: 1 to 335 disclosed in the associated sequence listing. Included with the present application is sequence listing comprising 340 sequences of antigens. For adenovirus target antigens include the hexon and penton.

For BK virus target antigens include large T antigen and small t-antigen.

In one embodiment the peptide mix comprises or consists of 2-1000 peptides, more specifically 2-500, for example 2-400, 2-300, 2-200 or 2-100 such as 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 53, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 347, 148, 149, 150, 151, 152, 153, 154 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199 or 200 peptides.

In one embodiment 5 to 500 ng of peptide or each peptide library are employed per mi of culture, such as 5, 50, 100, 150, 200, 250, 300, 350, 400 or 450 ng/ml, in particular 5 ng/ml. Alternatively 1, 2, 3 or 4 ng/mi of peptide may be employed.

Advantageously, peptides employed at a concentration of about 5 ng/ml (such as 4, 5, 6, 7, 8, 9 or 10 ng/ml) may result in an expanded antigen specific T cell population that at day 9 or day 10 secrete interferon gamma at levels equivalent or higher to cells expanded employing higher concentrations of peptides.

In one embodiment the peptides employed are GMP grade. That is to say they are manufactured using good manufacturing practice, which means they are suitable for use for the preparation of a therapeutic product.

Cytokines that may be employed in the process of the current disclosure include IL-1, IL-2, IL-4, IL-6 IL-7, IL-12 and IL-15.

A large amount of, as yet non-definitive, literature underlines how IL-2, IL-7 and IL-15 play non-redundant roles in shaping the representation of memory cells. IL-2 controls T-cell clonal expansion and contraction, and promotes lymphocyte differentiation. IL-2 and IL-15 can also support memory cell division and have been used in combination with antigen-driven stimulation, for the expansion of CTL. IL-7 regulates peripheral T cell homeostasis, and contributes to the generation and long-term survival of both CD4 and CD8 memory T lymphocytes in vivo.

In one embodiment the cytokines employed in the process according to the present disclosure are independently selected from IL-4, IL-7, and IL-15, especially IL-4 and IL-7.

In one embodiment the cytokines employed are IL-4 and/or IL-7. Whilst not wishing to be bound by theory the inventors believe that these cytokines have a role to play in shaping the frequency, repertoire and expansion of viral antigen-specific T ceils.

The repertoire of T ceils may be determined by ELISPOT analysis after stimulation with peptide libraries aliquoted into pools such that each peptide is uniquely represented in two pools (Kern, F., N. Faulhaber, C. Frommel, E. Khatamzas, S. Prosch, C. Schonemann, I. Kretzschmar, R. Volkmer-Engert, H. D. Volk, and P. Reinke. 2000. Analysis of CD8 T cell reactivity to cytomegalovirus using protein-spanning pools of overlapping pentadecapeptides. Eur J Immunol. 30:1676-1682 and Straathof, K. C., A. M. Leen, E. L. Buza, G. Taylor, M. H. Huls, H. E. Heslop, C. M. Rooney, and C. M. Bollard. 2005.

Characterization of latent membrane protein 2 specificity in CTL lines from patients with EBV-positive nasopharyngeal carcinoma and lymphoma. J. Immunol. 175:4137-4147).

IL-4 is generally employed at a final concentration of 250 ng/ml of culture or less, such as 200 ng/ml or less.

IL-7 is generally employed at a final concentration of 50 ng/ml of culture or less, such as 20 ng/ml or less, in particular 10 ng/ml.

If IL-15 is employed a suitable final concentration is 50 ng/ml of culture or less, such as 20 ng/ml or less, in particular 10 ng/ml.

In one embodiment in about 20 mls per GRex-10) (for example 20×10⁶ PBMCs) a further 10 mls medium containing IL-4 (1666 units per ml) and IL-7 (10 ng per ml) is added.

IL-12 has a role in Th1 focusing and exogenous IL-12 may be omitted if a balanced Th1/Th2 is desired. In one embodiment the process of the present disclosure does not employ exogenous IL-12. However, in the context of the present T cell product a Th1 response in the CD4+ population is thought to be desirable.

In one embodiment when IL-4 is employed the in the expansion process of the present disclosure at day 10 or day 11 the number of expanded cells may be 10, 20, 30, 40 50, 60, 70, 80, 90, 100 or 200% higher than cells expanded employing a similar protocol replacing IL-4 with IL-2.

However, ever where after about 9, 10 or 11 days the number of antigen specific T cells provided from the IL-2 and IL-4/7 protocols are the same in terms of number of specific gamma secreting cells the IL-2 cells are much less viable. Cell viability may be tested as described herein.

When exogenous IL-2 is employed in the rapid expansion system hyper-proliferation of T cells is generated. When this hyper-rapid expansion occurs then the balance of desirable T cells and the residual cells is suboptimal in that the expansion happens so rapidly that many that the residual cells have not died and thus remain present in the total cell population. Thus the present inventors have reconciled the inherently incompatible factors of rapid expansion with the selectivity of culturing the cells for a period of time and have found that the omission of IL-2 improves the ratio of desired cells to residual cells. What is more in the period 7 to 14 days such as 10 days the ratio of desired cells to residual cells is a cross-over point where the cultured product becomes suitable for use in therapy. This cross-over point is defined as when a sufficient minimum dose of therapeutic T cells is achieved within a dose formulation which falls within the safety threshold of no more than 5×10⁵ CD3+ T cells per kg of patient body weight.

It is expected by the inventors that this safety threshold will become the gold standard within the context of the optimal dosing for an antigen-specific T cell product aimed at providing immune reconstitution following bone marrow transplantation.

Whilst each final expanded T cell product may vary in the exact composition due to inherent differences in the starting donor sample the safety profile of the product can be controlled by monitoring the proportion of antigen specific T cells to residual cells. Advantageously, the product obtained from the process of the present invention can be controlled to ensure it is safe, in a clinical context.

In one embodiment the expanded antigen-specific T cell population obtained is biased to producing a CD4+ cell population.

In one embodiment the process of the present disclosure is employed to provide a cell population comprising a CD4+ T cell population, for example a Th1 population. A Th1 population as employed herein is intended to refer to a CD4+ population wherein 5% of the cells or more, such as 10, 20, 30, 40, 50, 60, 70, 80, 90% or more are classified as Th1.

Memory T cells are a component of Th1 cells.

In one embodiment the population of cells obtained from the process comprises a sub-population of memory T cells, for example the memory T cells represent 10, 20, 30, 40, 50 or 60% of the expanded cells and will generally express effector memory markers including CD27, CD28, CD62L and CD45RO. This will be a significantly higher than the population of memory cells prior to expansion.

Target antigen as employed herein is intended to refer to the antigen which is employed to the generate specificity in the T cells to the therapeutic target, such as a particular virus, for example CMV, adenovirus, EBV or BK Virus. Thus the cells infected by target virus or cancer cells will usually express the target antigen and hence will themselves become a target for clearance by the immune system. The immune system has the extraordinary capability to remove (kill) an infected cell from the body while ensuring minimal escape opportunities for the pathogen infecting the cell in question.

Residual CD3−, CD56+, and NK cells in the final cell population are acceptable since these are potentially beneficial.

The cell populations expanded using the process of the present disclosure comprises the desired T cell population and generally will not consist only of the desired population. The final product administered to the patient will include a number of other cells that the process did not target the expansion of. In one embodiment the desired population of CD4+ and CD8+ cells comprises about 60% or less, 70% or less, or 80% or less of the total population of cells. Frequency of the cell populations may be measured employing a gamma-IFN Elispot assay which is known to persons skilled in the art.

In one embodiment the T cells population obtained from the process are diverse when analysed by spectratyping, and without the emergence of dominant clone. That is to say the T cell diversity in the starting sample is substantially represented in the expanded T cells, i.e. the expansion is not generally the expansion of a single clone.

In one embodiment the relevant population of T cells prepared has a T cell receptor on the surface. In one or more embodiments the cell populations according to the present disclosure have one or more advantageous properties in comparison to cells prepared by the prior art method.

In one embodiment the antigen specific T cells of the present disclosure have an average cell diameter which is 95% or less, for example 90% or less, such as 85% or less, more specifically 80% or less of the maximum cell diameter.

In one embodiment the average cell diameter of cells in the relevant T cell population is in the range 10 to 14 microns and the average cell diameter is about 10, 11, 12, 13 or 14 microns.

We believe sufficient cells even for the highest doses can be prepared employing the method of the present disclosure.

Release Criteria for the product includes:
Identity: Greater than 50% CD3+ of CD45+ cells
Viability: Greater than 70% of CD45+ cells
Safety: CD3+ Dose not exceed specify number of T cells
Virus Specific: Minimum 100 Virus specific Cells per Kg defined by specific IFNg production.

Advantageously the cells cultures of the present invention exhibit generally low toxicity, for example are associated with few toxicity intolerance responses, for example inflammatory responses, cell damage, flu like symptom, nausea, hair less or the like.

In some embodiments the cell populations according to the present disclosure may also provide one or more the following advantageous properties, for example levels of interferon gamma secretion, in vivo proliferation, up-regulation of a T cell activation marker (for example T cell receptors) may be high relative to the total number of antigen specific T cells in the population,

In one embodiment gamma-capture may be used to select a target cell population.

High levels of interferon gamma secretion as employed herein are intended to refer to the fact that on average (for example expressed as a mean) cells in a populations prepared by the current method may secrete higher levels of interferon gamma than cells prepared by prior art methods. In assaying this attribute, it may be necessary to rest the cells tor a period after expansion before measuring the level of interferon gamma secretion. Although this is can be measured at the population level—when measured at the single cell level it will reveal that a higher percentage of cells will be antigen specific—i.e. producing interferon-gamma compared with cells made using the prior art.

In one embodiment the cells of the present disclosure may show enhanced antigen specificity, for example in an assay disclosure herein, in comparison to cells prepared by a prior art method.

In one embodiment the cell populations of the present disclosure show comparable avidity (not significantly different) to cell populations prepared by a prior art method.

The therapeutic antigen-specific T cell population provided may technically be a sub-therapeutic dose in the composition of the invention. However, after infusion into the patient the cells expand further to assist in reconstituting the patients' immune repertoire. Thus if the ceils transfused are exhausted or anergic then the effectiveness of the cells in vivo is likely to compromised or reduced in one or more respects.

Thus “potency” or “biological effectiveness” of the cells is a really important consideration in the reconstitution abilities of the target therapeutic T cells.

The present inventors believe that this insight will lead to a paradigm shift in the current thinking and approach to T cell therapy in the field.

In one embodiment the T cell populations provided by the present disclosure are effective in expanding in vivo to provide an appropriate immune response to cells infected by a target virus and/or cancer cells associated with a target virus.

The present invention also extends to compositions comprising the allogeneic antigen specific T cell populations according to the invention. These compositions may comprise a diluent, carrier, stabilizer, surfactant, pH adjustment or any other pharmaceutically acceptable excipient added to the cell population after the main process steps. An excipient will generally have a function of stabilizing the formulation, prolonging half-life, rendering the composition more compatible with the in vivo system of the patient or the like.

In one embodiment a protein stabilising agent is added to the cell culture after manufacturing, for example albumin, in particular human serum album, which may act as a stabilizing agent The amounts albumin employed in the formulation may be 1 to 50% w/w, for example 10 to 50% w/w, such as about 2.25, 4.5 or 12.5% w/w.

In one embodiment the formulation also contains a cryopreservative, for example glycerol or DMSO. The quantity of DMSO is generally 12% or less such as about 10% w/w.

In one embodiment the process of the present invention comprises the further step of preparing a pharmaceutical formulation by adding a pharmaceutically acceptable excipient, in particular an excipient as described herein, for example diluent, stabilizer and/or preservative.

Excipient as employed herein is a generic term to cover all ingredients added to the T cell population that do not have a biological or physiological function.

In one aspect there is provided a pharmaceutical composition of an allogeneic expanded antigen specific T cell population for a patent, wherein the composition comprises:
a population of CD3+ T cells, where in the total population of CD3+ does not exceed 5×10⁵ cells per Kg of patient, and said population of CD3+ T cells composes a therapeutic T cell population of CD4+ cells characterisation as a Th1 population, for example with some CD8+ CTLs, optionally the population of non-CD3+ cells is 20 percent or less of the total cell population of the composition, characterised in that the relevant expanded antigen-specific T cell population is able to expand in vivo.

In one embodiment there is provided a pharmaceutical composition expanded antigen-specific T cell population for a patient, wherein the composition comprises:
a population of at least 70% CD3+ T cells and less than 30% of non CD3+ cells, where the total population of CD3+ cells does not exceed 5×10⁴ cells (or alternatively 1×10⁵) per Kg of patient, and said population of CD3+ T cells comprises a therapeutic T cell population of CD4+ cells which have a primary characterization as Th1,
   characterised in that the relevant expanded antigen specific T cell population is able to expand in vivo.

Analysis of whether the antigen specific T cells are suitable for expanding in vivo may be tested employing in vitro systems, for example using a cell proliferation assay, for example the CFSE assay described herein.

In one embodiment the expanded antigen specific T cells are capable of further expansion in vitro and in vivo, significant levels expansion for example include 2, 3, 4, 5 fold expansion or more.

In one embodiment at least 70% of the relevant cells are viable as measured by dye exclusion or flow cytometry, for example 75%, 80%, 85% or more of the cells are viable.

In one embodiment the expanded antigen specific T cell population are capable of producing Th1 cytokines, for example biologically relevant levels thereof.

Cell proliferation may be assayed by labelling cells with fluorescent compound CFSE to monitor division to a given stimulus, In short cells are labelled with CFSE and antigen is added which stimulates some cells to divide. These cells can be monitored as when they divide the amount of dye in each daughter cell is halved thus halving the brightness of the cell as detected by flow cytometry. Therefore the number of divisions the cell population has undergone can be determined.

Once the final formulation has been prepared it will be filled into a suitable container for example an infusion bag or cryovial.

In one embodiment the process according to the present disclosure comprises the further step of filling the T cell population or pharmaceutical formulation thereof into a suitable container, such as an infusion bag and sealing the same.

In one embodiment the container filled with the T cell population of the present disclosure or a pharmaceutical composition comprising the same is frozen for storage and transport, for example is store at about −135° C.

In one embodiment the process of the present disclosure comprises the further step of freezing the T cell population of the present disclosure or a pharmaceutical composition comprising the same. In one embodiment the “product” is frozen by reducing the temperature by 1° C. per minute to ensure the crystals formed do not disrupt the cell structure. This process may be continued until the sample has reached about −100° C.

A product according to the present disclosure is intended to refer to a cultured cell population of the present disclosure or a pharmaceutical composition comprising the same.

In one embodiment the product is transferred, shipped, transported in a frozen farm to the patient's location.

In one embodiment the product according to the present disclosure is provided in a form suitable for parenteral administration, for example, infusion, slow injection or bolus injection. In one embodiment the formulation is provided in a form suitable for intravenous infusion.

In one aspect the present disclosure provides a method of transport a product according to the present disclosure, from the place of manufacture, or a convenient collection point to the vicinity of the intended patient, for example where the T cell product is stored at or below 0° C. during transit, such as below −100° C.

In one embodiment the temperature fluctuations of the T cell product are monitored during storage and/or transport.

In one embodiment there is provided a product of the present disclosure for use in treatment, for example in the treatment of a viral pathogen such as adenovirus, CMV, EBV, human polyoma virus, herpes simplex virus, varicella zoster virus, hepatitis, rotavirus or similar.

In one embodiment the treatment is of an immunosuppressed patient.

In one embodiment there is a provided a method of treating a patient with a product according to the present disclosure comprising the step of administering a therapeutically effective amount of product defined herein.

Therapeutically effective amount does not necessarily mean an amount that is immediately therapeutically effective but includes a dose which is suitable for expansion in vivo (after administration) to provide a therapeutic effect.

It is envisaged that more than one embodiment described herein may be combined, as technically appropriate.

In the context of this specification “comprising” is to be interpreted as “including”.

Aspects of the disclosure comprising certain elements are also intended to extend to alternative embodiments “consisting” or “consisting essentially” of the relevant elements.

All references referred to herein are specifically incorporated by reference,
Sili U et al Large-scale expansion of dendritic cell-primed polycolonal human cytotoxic T-lymphocyte lines using lymphoblastoid cells for adoptive immunotherapy. J. Immuother 2003 May-June: 26(3): 241-56.
Leen A M et al Contact-activated monocytes: efficient antigen presenting cells for the stimulation of antigen-specific T cells. J. Immunother. 200 7 January: 30(1): 96-107.
Bollard C M et al The generation and characterization of LMP2-specific CTL for use as adoptive transfer from patients with relapsed EBV-positive Hodgkin disease J. Immunother.
MG Wing, et al An Improved Method for the Detection of Cell Surface Antigens in Samples of Low Viability using Flow Cytometry. J Immunol Methods 326:21-27, 1990.
D R Parks, et al Chapter 29 Flow Cytometry and Fluorescence-Activated Cell Sorting (FACS). Handbook of Experimental Immunology, DM Weir(ed), Blackwell Scientific Publications, MA, 1986.
I. Schmid et al Dead Cell Discrimination with 7 aminoactinomycin D in Combination with Dual Colour Immunofluorescence in Single Laser Flow Cytometry. Cytometry 13:204, 1992.
F. deBoer et al Extensive early apoptosis in frozen thawed CD34+ stem cells decreases threshold doses for haematological recovery after autologous peripheral blood progenitor cell transplant. Bone marrow Transplant 29:249-255, 2002.
RS Anthony, et al Flow cytometry using annexin V can detect early apoptosis in peripheral blood stem cell harvests from patients with leukemia and lymphoma. Bone Marrow Transplant 21:441-446, 1998. James W Tung, et al. Modern Flow Cytometry: A Practical Approach. Clin Lab Med 27(3):453, 2007 (September).

### EXAMPLES

### Expansion of the Antigen Specific T Cell Product Reagents

This procedure describes the production and cryopreservation of an adoptive cellular therapy, produced using a Wilson Wolfe GRex system:

| Reagents | Manufacturer | Catalogue No. |
|---|---|---|
| RPMI 1640 | Invitrogen Ltd | 61870 |
| Human AB serum | | |
| DMSO | Wak chemie | WAK-DMSO-70 |
| Lymphoprep | AxisSheild | 11114445 |
| Human Serum Albumin (HSA), 4.5% | BPL | PL08801/0006 |
| IL-4 | CellGenix | 1003 |
| IL-7 | CellGenix | 1010 |
| AdV-5 | Miltenyi | 76106 |
| WFI | Gibco | A12873 |

A peptide or peptide mix from the target virus such as CMV pp65 or PepTivator®:

| EQUIPMENT | Manufacturer | Specifications |
|---|---|---|
| Centrifuge Rotina 46R | Not Specified | 300 × ‘g’ |
| Digital Balance | Not Specified | Weight |
| Plasma Press | Baxter | N/A |
| CO₂ Incubator | Not Specified | 37 ± 2° C. 5 ± 2° C. |
| Heat Sealer | Baxter | N/A |
| Sterile Welder | Terumo | N/A |
| Class II Biological safety cabinet | Not Specified | N/A |
| Kryo 560-16 | Planer | TBC |
| Vacuum chamber | Multivac | N/A |
| Sepax Blood Processor | Biosafe | N/A |
| Micropipettes | Gilson | N/A |

### Incoming Blood Product Receipt:

All donors must be screened for Markers of Infectious Diseases (Hepatitis B surface antigen, Hepatitis C, Syphilis, HTLV I and II and HIV markers) within 30 days of the donor blood product extraction. Should a donor have tested positive for any infectious disease, the product collection will not be carried out and the processing cancelled.

### IL-4

Dilute a 50 μg vial of IL-4 with 250 μl of Water For Injection, producing a 200 μg/ml stock solution. 20 μL of diluted sample is added per culture when required. The remaining sample is aliquoted into 50 μL aliquots in 1.8 ml Nunc Cryovials. If using a frozen batch of IL-4 ensure the material is fully thawed before use.

### IL-7

Dilute 50 μg vial of IL-7 with 200 μl of Water For Injection (WFI), The solution is further diluted 1:25, by adding 240 μl of WFI to produce a 10 μg/ml working stock. 20 μl of this is then used per culture.

The remaining sample is aliquoted into 50 μl aliquots in 1.8 ml Nunc Cryovials. If using a frozen batch of IL-7 ensure the material is fully thawed before use.

### Peptide

The peptide is reconstituted by adding 2 ml of WFI into the 100 μg/peptide vial. 100 μl of the reconstituted peptide is removed and further diluted in 900 μl of WFI. 20 μL of the diluted peptide is then added to each culture. 50 μl aliquots of the reconstituted peptide are dispensed into Nunc cryovials. The remaining 1500 μl of concentrated peptide (50 μg/ml) is released for use in the QC assays.

### RPMI for Washing

Spike a 5 L bag of RPMI the coupler from a 600 ml transfer pack. Place the transfer pack onto a tared balance and transfer Approximately 500 ml (500 g) of RPMI. Once all the buffer has been transferred heat seal the line three times. Lebel as ‘Patient Identifier, 500 ml for washing and leave at 2-8° C. until required far use.

### RPMI for Inoculation

Weld on a fresh 600 ml transfer pack onto the remaining tubing connected to the 5 L bag of RPMI, using a tared balance transfer approximately 100 ml of RPMI into the bag.

### Lymphoprep

Spike a bottle of Lymphoprep with the coupler from a 1000 ml bag. Spike the Lymphoprep with two air inlets and transfer 100 ml of into the bag. Label as ‘Patient Identifier, 100 ml Lymphoprep.

### Mononuclear Cells Preparation-Day 0

PBMC are prepared by density gradient centrifugation on the sepax device, a protocol known to those skilled in the art.

### Example 1 Process for 226 CMV

### Day-0

### Buffer Preparation

### IL-4

A 50 μg vial of IL-4 (USP grade, CellGenix cat 1003-050) was diluted with 250 μL of WFI (USP Grade Invitrogen Cat A12873) to produce a 200 μg/ml stock solution. The stock solution was stored at −80° C. with an aliquot being left for use in the pot inoculation.

### IL-7

A 50 μg vial of IL-4 (GMP, Celigenix cat 1010-050) was diluted with 200 μL of WFI (USP Grade Invitrogen Cat A12873) to produce a stock. The stock was diluted 1:25 with WFI to produce a working stock, which was then stored at −80° C. with an aliquot being left for use in the pot inoculation.

### CMV Peptivator Peptide

A 60 nmol/peptide peptide (GMP PepTivator pp65) was reconstituted in 2 ml of WFI (USP Grade Invitrogen Cat A12873). 100 μl of the reconstituted peptide was removed and further diluted in 900 μL of WFI. 20 μL of the diluted peptide was retained for pot inoculation with the remaining volume aliquoted and stored at −80° C.

### RPMI

A 5 L bag of RPMI+Glutamax (Invitrogen Cat 61870) was connected to a 600 ml transfer pack and 500 ml of RPMI transferred (product washing). The RPMI was welded onto a 2^{nd} 600 ml transfer pack and a further 100 ml transferred (pot inoculation).

### Lymphoprep

100 ml of Lymphoprep (Axis Shield Cat 1114740) was drained into a 1000 ml transfer pack and set aside for use within the DGBS (Density gradient based separation) cycle

### Cell Manipulation.

100 ml of non-mobilised aphaeresis arrived on site, after a temperature monitored shipment at 2-8° C., from a matched donor. A 3 ml sample (STA) of the cell product was removed and 1 ml inoculated into a set of Bactecs. The following QC and in-process testing was also performed on the remaining starting material with the results displayed in the table below.
[symbol] Cell Count, performed on automated cell counter
Absolute T cell Enumeration via Trucount (CD3-FITC, CD8-PE, CD45-PerCP)
Viability Stain (CD3-FITC, CD45-PE, CD8-PerCP)

| WBC Count | 34.4 × 10⁶/ml |
|---|---|
| Lymphocyte % | 80.5% |
| Absolute CD3 Conc | 21.6 × 10⁶ |
| Viability | 98.87% |
| Haematocrit | 1.6% |

The 500 ml bag of RPMI and 100 ml of Lymphoprep were connected to the CS900.02 along with the remaining contents of the incoming whole blood materials (97 ml). The V128 DGBS cycle was selected on the Sepax and the kit installed. On completion of the cycle 42 ml of PBMC's were eluted and the following testing performed.

A 1 ml sample of removed from the elution bag and the following tests were performed
Cell Count, performed on automated cell counter
Absolute T cell Enumeration via Trucount (CD3-FITC CD8-PE, CD45-PerCP)
[symbol] Viability Stain (CD3-FITC, Cd45-PE, CD8-PerCP)

| WBC Count | 48.0 × 10⁶/ml |
|---|---|
| Lymphocyte % | 79.2% |
| Absolute CD3 Conc | 20.66 × 10⁶/ml |
| Viability | 97.58% |

20×10⁶ WBC's (410 μl of cell suspension) were inoculated into the Wilson Wolfe Biopot (GP-40 Bioreactor) along with 2 ml of Human AB serum (GMP German Blood Service) and 20 μl of the IL-4/IL-7 and peptide. The total volume is made up to 20 ml using RPMI (19.6 ml).

The pot was then incubated for 10 days in a 37±2° C., 5±1% CO₂, 95% humidity incubator.

### Day-10

### Cell Washing

On day 10 the pot is removed from the incubator and gently agitated to resuspend the cells from the base of the Wilson Wolfe biopot. A 2 ml sample is removed with 2×250 μL being using for Mycoplasma testing. The remaining sample is used in the following in process testing:
[symbol] Manual cell count with Trypan Blue
[symbol] T Cell Identity CD3-FITC, CD56-PE, CD45-PerCP

| Volume Recovered | 12.5 ml |
|---|---|
| WBC Cell Count | 3.6 × 10⁶ |
| Viability | 73% |

A 230 ml bag of 45% HAS (BPL) was prepared and the remaining cell suspension was diluted before being spun at 300×‘g’ for 10 minutes. On completion of the spin the cell pack the supernatant is removed using a plasma press before the cells are resuspened and diluted in a further 230 ml of 4.5% HSA. The cells are given a final spin at 300×‘g’ for 10 minutes before the supernatant is again removed. The pellet is resuspended in fresh 4.5% HSA.

A 1 ml sample is removed and the following QC and in process testing performed
[symbol] Intra-cellular gamma stain

| | |
|---|---|
| IFN-Gamma + T cells | 15.07% |

### Freezing and Shipping

100 ml of 20% DMSO (Cryosure-DMSO, Wak-Chemie) solution IN 4.5% HSA (BPL) was prepared and chilled on ice packs
The cell dose required is =3×10⁶ T cells/kg
Patient Weight=16 kg
Therefore Cells Required=480,000 T cells

The following volumes were transferred into a suitably sized Cryocyte bag via a Cryocyte manifold set.

| | |
|---|---|
| Cell Suspnsion | 0.13 ml |
| 4.5% has | 9.81 ml |
| 20% DMSO in 4.5% HSA | 10.0 ml |

A 3 ml sample was removed from the Cryocyte bag with 2 ml being inoculated into one of each of a set of Bactecs The remaining 1 ml of sample was stored at −80° C. and sent for Endotoxin testing.

The Cryocyte bag containing the cell product was placed within a controlled rate freezer and the cells frozen at a rate of −1° C./min until −30° C. and then reduced at −2° C./min to −100° C. On completion of the freezing cycle the cells were placed with the vapour phase of a temperature monitored LN₂ Dewar. The following safety testing results were obtained prior to product release.

| | |
|---|---|
| Sterility | No growth detected |
| Mycoplasma | Negntive |
| Endotoxin | ≤10 EU/ml |

Once requested by the patients physician the product was released by the QP before being shipped to the relevant stem cell labs within a validated, temperature monitored dry shipper. Upon arrival the cells were stored within the vapour phase of the stern cells labs dewars until infusion.

### Example 2 Process for 226 ADV

### Day-0

### Buffer Preparation

### IL-4

A 50 μg vial of IL-4 (USP grade, CellGenix cat 1003-050) was diluted with 250 μL of WPI (USP Grade Invitrogen Cat A12873) to produce a 200 μg/ml stock solution. The stock solution was stored at −80° C. with an aliquot being left for use in the pot inoculation.

### IL-7

A 50 μg vial of IL-4 (GMP, Cellgenix cat 1010-050) was diluted with 200 μL of WFI (USP Grade Invitrogen Cat A12873) to produce a stock. The stock was diluted 1:25 with WFI to produce a working stack, which was then stored at −80° C. with an aliquot being left for use in the pot inoculation.

### ADV Peptivator Peptide

A 60 nmol/peptide peptide (GMP PepTivator hexon V) was reconstituted in 2 ml of WFI (USP Grade Invitrogen Cat A12873). 100 μl of the reconstituted peptide was removed and further diluted in 900 μl of WFI. 20 μL of the diluted peptide was retained for pot inoculation with the remaining volume aliquoted and stored at −80° C.

### RPMI

A 5 L bag of RPMI+Glutamax (Invitrogen Cat 61870) was connected to a 600 ml transfer pack and 500 ml of RPMI transferred (product washing). The RPMI was welded onto a 2^{nd} 600 ml transfer pack and a further 100 ml transferred (pot inoculation).

### Lymphoprep

100 ml of Lymphoprep (Axis Shield Cat 1114740) was drained into a 10 1000 ml transfer pack and set aside for use within the DGBS (Density gradient based separation) cycle.

### Cell Manipulation

100 ml of non-mobilised aphaeresis arrived on site, after a temperature monitored shipment at 2-8° C., from a matched donor. A 3 ml sample (STA) of the cell product was removed and 1 ml inoculated into a set of Bactecs. The following QC and in-process testing was also performed on the remaining starting material with the results displayed in the table below.
[symbol] Cell Count, performed on automated cell counter.
[symbol] Absolute T cell Enumeration via Trucount (CD3-FITC, CD8-PE, CD45-PerCP).
[symbol] Viability Stain (CD3-FITC, CD45-PE, CD8-PerCP).

| WBC Count | 34.4 × 10⁶ ml |
|---|---|
| Lymphocyte % | 80.5% |
| Absolute CD3 Conc | 21.6 × 10⁶ |
| Viability | 93.87% |
| Haematocrit | 1.6% |

The 500 ml bag of RPMI and 100 ml of Lymphoprep were connected to the CS900.02 along with the remaining contents of the incoming whole blood materials (97 ml). The V128 DGBS cycle was selected on the Sepax and the kit installed. On completion of the cycle 42 ml of PBMC's were eluted and the following testing performed.

A 1 ml sample of removed from the elution bag and the following tests were performed.
[symbol] Cell Count, performed on automated cell counter.
Absolute T cell Enumeration via Trucount (COS-FITC, CD8-PE, CD45-PerCP).
Viability Stain (CD3-FITC, Cd45-PE, CD8-PerCP).

| MC Count | 48.0 × 10⁶ ml |
|---|---|
| Lymphocyte % | 79.2% |
| Absolute CD3 Conc | 20.66 × 10⁶ ml |
| Viability | 91.58% |

20×10⁵ WBC's (410 μl of cell suspension) were inoculated into the Wilson Wolfe Biopot (GP-40 Bioreactor) along with 2 ml of Human AB serum (GMP German Blood Service) and 20 μl of the IL-4/IL-7 and peptide. The total volume is made up to 20 ml using RPMI (19.6 ml).

The pot was then incubated for 10 days in a 37±2° C., 5±1% CO₂, 95% humidity incubator.

### Day-10

### Cell Washing

On day 10 the pot is removed from the incubator and gently agitated to resuspend the cells from the base of the Wilson Wolfe biopot. A 2 ml sample is removed with 2×250 μl being using for Mycoplasma testing. The remaining sample is used in the following in process testing.
Manual cell count with Trypan Blue.
T Cell Identity CD3-FITC, CD56-PE, CD45-PerCP.

| Volume Recovered | 12.5 ml |
|---|---|
| WBC Cell Count | 3.6 × 10⁶ |
| Viability | 97.22% |

A 230 ml bag of 4.5% HAS (BPL) was prepared and the remaining cell suspension was diluted before being spun at 300×‘g’ for 10 minutes. On completion of the spin the cell pack the supernatant is removed using a plasma press before the cells are resuspended and diluted in a further 230 ml of 4 5% HSA. The cells are given a final spin at 300×‘g’ for 10 minutes before the supernatant is again removed. The pellet is resuspended in fresh 4.5% HSA.

A 1 ml sample is removed and the following QC and in process testing performed.
Intra-cellular gamma stain

### Freezing and Shipping

100 ml of 20% DMSO (Cryosure-DMSO, Wak-Chemie) solution IN 4.5% HSA (BPL) was prepared and chilled on ice packs.
The cell dose required is =3×10⁴ Tcells/kg.
Patient Weight=16 kg.
Therefore Cells Required=480,000 T cells.

The following volumes were transferred into a suitably sized Cryocyte bag via a Cryocyte manifold set.

| | |
|---|---|
| Cell Suspension | 0.13 ml |
| 4.5% HSA | 9.87 ml |
| 20% DMSO in 4.5% HSA | 10.0 ml |

A 3 ml sample was removed from the Cryocyte bag with 2 ml being inoculated into one of each of a set of Bactecs. The remaining 1 ml of sample was stored at −80° C. and sent for Endotoxin testing.

The Cryocyte bag containing the cell product was placed within a controlled rate freezer and the cells frozen at a rate of −1° C./min until −30° C. and then reduced at −2° C./min to −100° C. On completion of the freezing cycle the cells were placed with the vapour phase of a temperature monitored LN₂ Dewar. The following safety testing results were obtained prior to product release.

| | |
|---|---|
| Sterility | No growth detected |
| Mycoplasma | Negative |
| Endotoxin | ≤10 EU/ml |

Once requested by the patients physician the product was released by the QP before being shipped to the relevant stem cell labs within a validated, temperature monitored dry shipper. Upon arrival the cells were stored within the vapour phase of the stem cells labs dewars until infusion.

### Example 3

The starting population of cells was cultured in an adapted G-Rex system as shown in the Figures employing RPMI 1640 media in the presence of 10% human serum, IL4, IL7 and an overlapping peptide pool specific for the desired antigen. For CMV specific expansion then the peptide employed was pp65. For adenovirus (ADV) overlapping peptides for the ad 5 hexon were employed. A seed density of 0.5, 1 or 2×10⁶/cm² was employed. The results established that 2×10⁶/cm² lead to maximal cell expansion see Table 1 Seeding Density for CMV expansion:

| | | Day 0 | Day 5 | Day 9 | Day 12 |
|---|---|---|---|---|---|
| Donor I | no cyto | 20000000 | 18300000 | 13500000 | 14100000 |
| | IL4/7 | 20000000 | 29100000 | 108000000 | 120000000 |
| | no cyto | 10000000 | 7800000 | 6900000 | 8100000 |
| | IL4/7 | 10000000 | 13500000 | 43500000 | 81000000 |
| | no cyto | 5000000 | 1800000 | 2100000 | 1900000 |
| | IL4/7 | 5000000 | 4200000 | 6400000 | 3900000 |
| Donor II | no cyto | 20000000 | 13500000 | 15000000 | 22800000 |
| | IL4/7 | 20000000 | 14700000 | 34500000 | 120000000 |
| | no cyto | 10000000 | 4500000 | 5500000 | 5000000 |
| | IL4/7 | 10000000 | 12500000 | 32000000 | 75000000 |
| | no cyto | 5000000 | 4200000 | 2100000 | 2700000 |
| | IL4/7 | 5000000 | 2700000 | 6300000 | 21000000 |
| Donor III | no cyto | 20000000 | 9900000 | 11100000 | 13500000 |
| | IL4/7 | 20000000 | 13500000 | 24000000 | 49000000 |
| | no Qyto | 10000000 | 5100000 | 6000000 | 5500000 |
| | IL4/7 | 10000000 | 10500000 | 12000000 | 24000000 |
| | no cyto | 5000000 | 2100000 | 3500000 | 3000000 |
| | IL4/7 | 5000000 | 2500000 | 4200000 | 4500000 |

The melange was cultured for 12 about 12 days at 37° C., without stirring or agitation.

Samples were taken daily and analysed or as required.

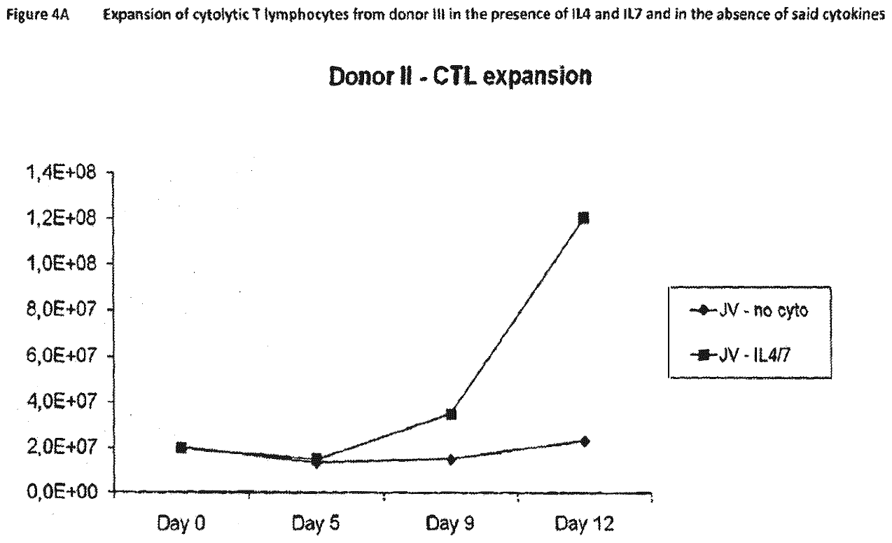

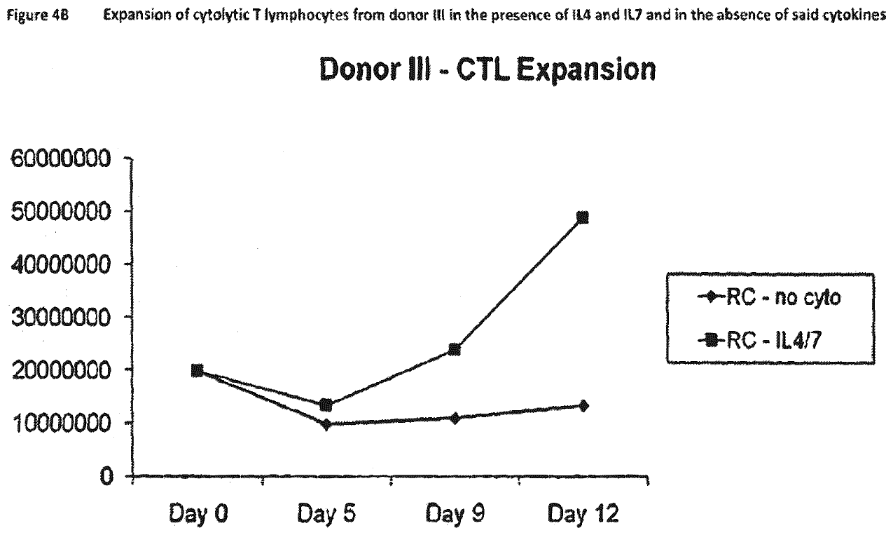

The total cell count of the expanded population was measured. The results for two donors are shown in FIGS. 4A and 4B.

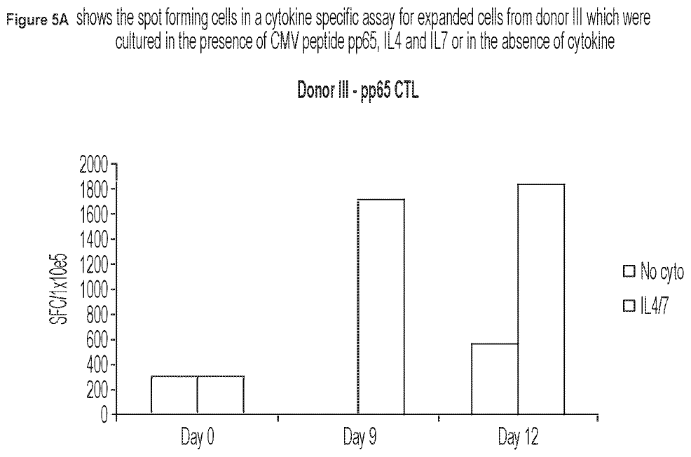

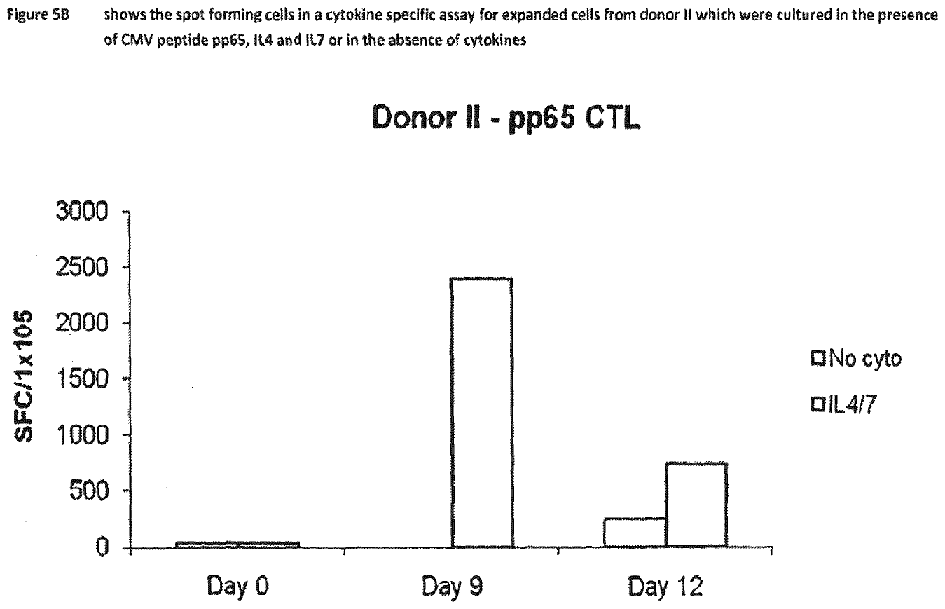

The amount of cells specific for CMV which secrete cytokines were tested. The results for two donors are shown in FIGS. 5A and 5B.

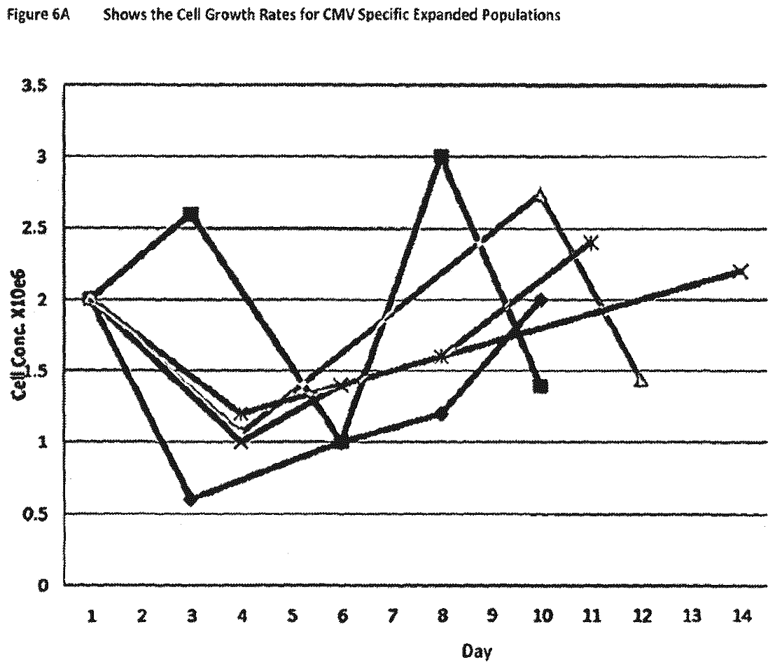

Cell growth rates were measures and results for CMV specific expansions are shown in FIG. 6A and for adeno specific expansions are shown in FIG. 6B.

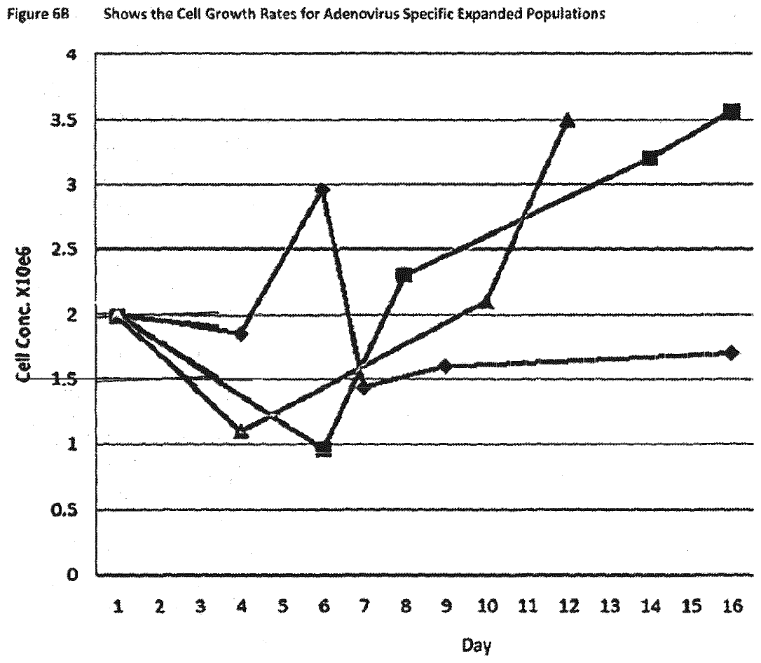

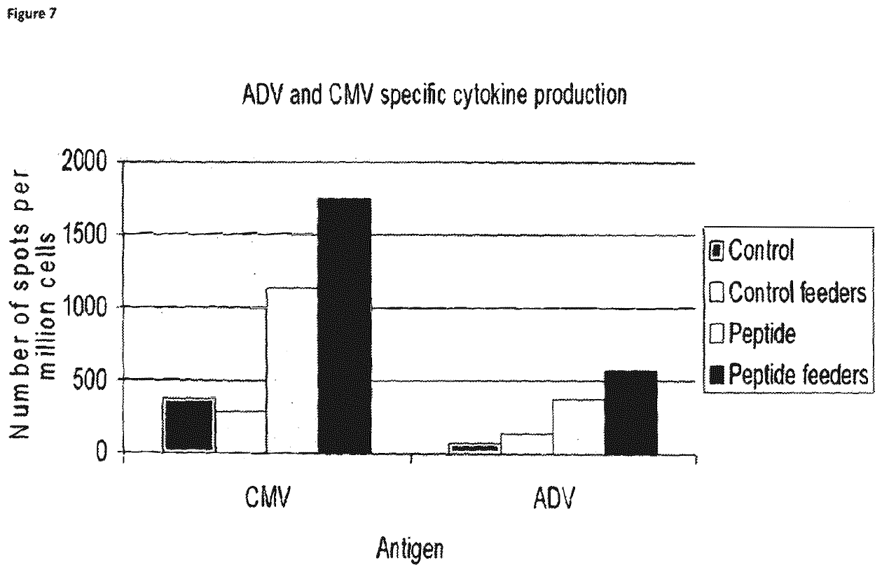

Adeno and CMV specific cytokine production was measured using elispot and the results are shown in FIG. 7.

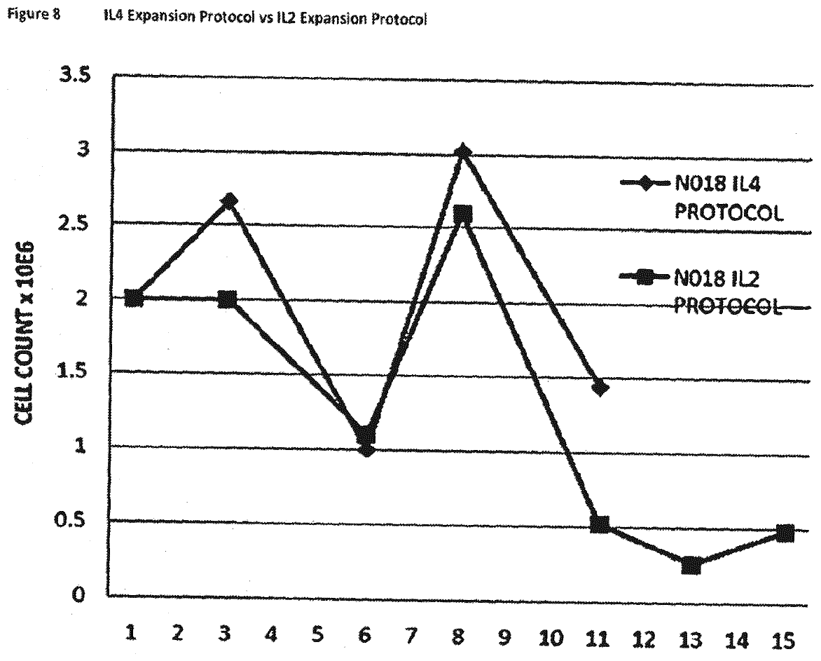

FIG. 8 shows a comparison of two expansion protocol, wherein the only cytokine employed is IL2 or IL4.

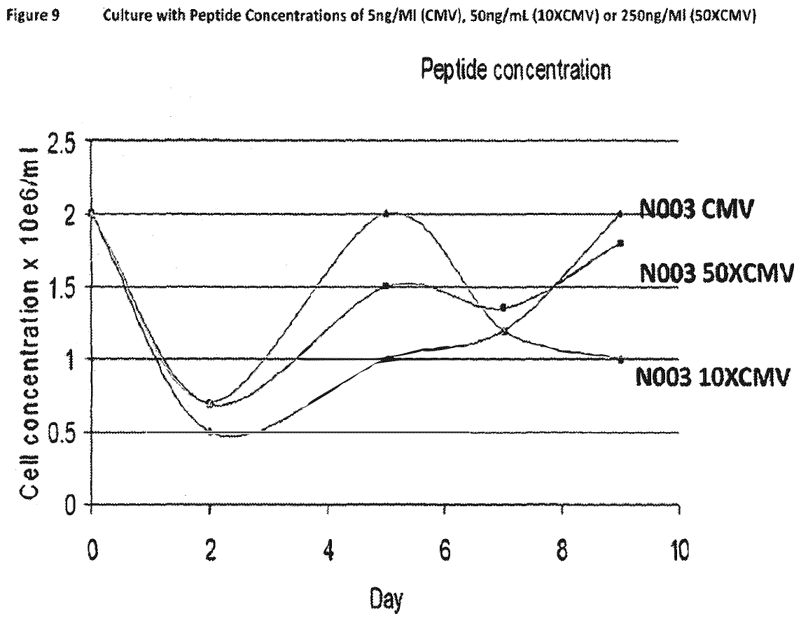

FIG. 9 show the expansion protocol of Example 7 employing different concentrations of peptides. At around day 9 or 10 the system employing 5 ng/ml (CMV) seems to have improved performance in terms of cell expansion.

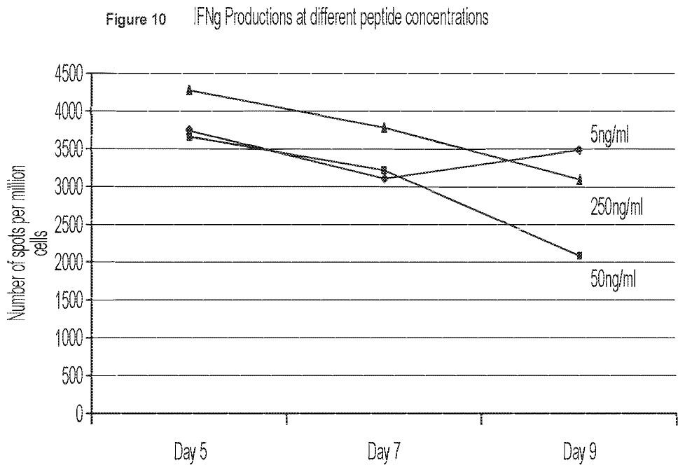

FIG. 10 shows IFN-gamma production of the cells cultured using different concentrations of peptide. At around day 9 or 10 the system employing 5 ng/ml (CM V) seems to perform best.

1. An antigen-specific T cell population (such as an allogeneic antigen specific T cell population) obtained or obtainable from a process for rapid expansion of antigen specific T cells comprising the steps culturing in a gas permeable vessel a population of PBMCs in the presence of a peptide or peptide mix relevant to a target antigen(s) in the presence or an exogenous cytokine characterized in that the cytokine is other than exogenous IL-2.

2. The antigen specific T cell population (such as an allogeneic antigen specific T cell population) according to claim 1, wherein no more than 30 percent of the cell are CD3−.

3. The antigen specific T cell population (such as an allogeneic antigen specific T cell population) according to claim 1, wherein the expansion is biased towards CD4+ cell expansion.

4. The antigen specific T cell population (such as an allogeneic antigen specific T cell population) according to claim 1, wherein the CD4+ T cell population is primarily a Th1 population.

5. A pharmaceutical composition of an expanded antigen specific T cell population for a patient, wherein the composition comprises: a population of at least 70% CD3+ T cells and less than 30% of non CD3+ cells.

6. The pharmaceutical composition of claim 5 wherein the total population of CD3+ cells does not exceed 5×10⁶ cells per Kg of patient.

7. The pharmaceutical composition of claim 5, wherein said population of CD3+ T cells comprises a therapeutic T cell population of CD4+ cells which have a primary characterization as Th1.

8. The pharmaceutical composition of claim 5, wherein the expanded antigen specific T cell population is able to expand in vivo.

9. The pharmaceutical composition according to claim 5 adapted for infusion.

10. The pharmaceutical composition according to claim 5, wherein the expanded antigen specific T cell population is at least 1% of the total cell population.

11. The pharmaceutical composition according to claim 5, wherein the expanded antigen specific T cell population is at least 5,000 cells.

12. The pharmaceutical composition according to claim 5, wherein the expanded antigen-specific cell population is in the range 20 to 80 percent of the CD3+ T cell population.

13. The pharmaceutical composition according to claim 5, wherein the expanded antigen specific T cell population is specific for a cell infected with CMV and/or presenting CMV antigens.

14. The pharmaceutical composition according to claim 5, wherein the expanded antigen specific T cell population is specific for a cell infected with adenovirus and/or presenting adenovirus antigens.

15. The pharmaceutical composition according claim 5, wherein the expanded antigen specific T cell population of the composition is expanded from a mobilized blood sample.

16. The pharmaceutical composition according to claim 5, wherein the cell population is cultured for 14 days or less, such as 12, 11 or 10 days.

17. The pharmaceutical composition according to claim 5 for use in the treatment or prophylaxis of a patient, for example a patient who has received a HS Cell transplantation.

18. The pharmaceutical composition according to claim 17, wherein the treatment or prophylaxis is for viral infection, for example CMV or ADV.

19. A method of treating a patient comprising administering a therapeutically effective amount of antigen specific T cell population as defined in claim 1.

20. A method of treating a patient comprising administering a therapeutically effective amount of a pharmaceutical composition according to claim 5.
